(19) United States
(12) Patent Application Publication
(10) Pub. No.: US 2021/0154634 A1
(43) Pub. Date: May 27, 2021

(54) REACTOR FOR PERFORMING EQUILIBRIUM-REDUCED REACTIONS

(71) Applicant: Siemens Aktiengesellschaft, Munich (DE)

(72) Inventors: Manfred Baldauf, Erlangen (DE); Lukas Glöser, Erlangen (DE); Katharina Meltzer, Erlangen (DE); Alexander Tremel, Möhrendorf (DE)

(73) Assignee: Siemens Aktiengesellschaft, Munich (DE)

(21) Appl. No.: 17/047,862

(22) PCT Filed: Apr. 17, 2019
(86) PCT No.: PCT/EP2019/059882

§ 371 Date: Oct. 15, 2020

(30) Foreign Application Priority Data
Apr. 20, 2018 (EP) 18168501.7

(51) Int. Cl. B01J 8/00; B01J 8/02; B01J 8/06

(52) U.S. Cl. CPC B01J 8/009; B01J 2208/00212; B01J 8/067; B01J 8/0221

(57) ABSTRACT

A reactor for performing equilibrium-reduced reactions, includes a tubular reactor housing in which a first zone is arranged, through which a liquid absorbent flows, and which extends in the longitudinal direction of the tube. Aa second zone is arranged for receiving a catalyst material and also extends in the longitudinal direction of the tube. The first zone and the second zone are separated by a gas-permeable separation zone. The separation zone has a mechanically self-supporting structure and the aspect ratio of the tubular reactor housing along a reaction zone is greater than 6.

### CROSS REFERENCE TO RELATED APPLICATIONS

This application is the US National Stage of International Application No. PCT/EP2019/059882 filed 17 Apr. 2019, and claims the benefit thereof. The International Application claims the benefit of European Application No. EP18168501 filed 20 Apr. 2018. All of the applications are incorporated by reference herein in their entirety.

### FIELD OF INVENTION

The invention relates to a reactor for implementation of equilibrium-reduced reactions, and to a reactor bundle, and to a method of operating a reactor, and to a power plant system comprising a power generator and a reactor.

### BACKGROUND OF INVENTION

Fossil energy carriers cause carbon dioxide emissions that make it difficult to achieve global climate protection targets. This is a driving force for the development of renewable energies. However, the generation of renewable power is subject to distinct fluctuations regionally and in terms of time.

For example, power is produced inexpensively on sunny or windy days by photovoltaic systems or wind turbines. There is currently a search for economic approaches to utilizing this power viably beyond the electricity sector and, for example, generating chemical products of value therefrom. One means of doing so is the electrochemical conversion of water to hydrogen and oxygen. The hydrogen generated can then react with carbon dioxide, which is harmful to the climate, as a starter molecule or reactant, which would simultaneously reduce carbon dioxide emissions. Carbon dioxide, which is relatively readily available, can thus react as an inexpensive carbon source, for example, in the preparation of methanol as a possible product of a one-stage synthesis of carbon dioxide and hydrogen according to the reaction equation

CO₂+3H₂->CH₃OH+H₂O.

A disadvantage of the synthesis of methanol from carbon dioxide and hydrogen is the lower equilibrium conversions that are, for instance, only 20% at 50 bar and 250° C. Therefore, in conventional synthesis plants, a major portion of the gaseous reactants is circulated, which leads to considerable pressure drops in the reactor and in pipelines, and which means that there has to be a considerable energy input in the form of compression output and heating output. Furthermore, gas recycling is not very suitable for dynamic operation of a reaction plant, and is therefore also difficult to match to volumes of electrical energy that occur irregularly, as is the prevailing case due to the fluctuating generation.

One approach to implementation of equilibrium-limited reactions is already described in WO 2017/212016 A1. This involves using a stirred tank reactor in which there is an absorbent in a lower region of the reactor, and reactant gases are guided through a catalyst arrangement, with absorption of products by absorbents far removed from the catalyst. Such a plant is certainly useful, but it is technically complex by virtue of the stirring arrangement and has limitations in the dynamic removal of the product and the dynamic supply of electrical energy.

US 2007/264 190 A1 discloses a reactor having a dynamic compression element that monitors a catalyst bed structure and hydrodynamic conditions within the reactor for physical or structural changes in the catalyst bed. A gas-permeable, hydrophobic membrane enables the separation of hydrogen from the catalyst bed.

WO 2017/162 410 A1 relates to a reactor for performance of equilibrium-limited reactions, comprising a reaction space for accommodation of a catalyst, and further comprising a sorption space suitable for accommodation of an absorbent. The reaction space and the sorption space are separated by an element that repels gas-permeable liquid droplets of the absorbent.

### SUMMARY OF INVENTION

It is an object of the invention to provide a reactor for implementation of equilibrium-limited reactions, which are achievable with a lower level of technical complexity compared to the prior art and permit a continuous process having good dynamic controllability.

The object is achieved by a reactor for implementation of equilibrium-limited reactions, by a reactor bundle, by a method of operating a reactor, and by a power plant system having a power generator described herein. The reactor of the invention for implementation of equilibrium-reduced reactions comprises a reactor housing 4 in tubular configuration. There is a first zone therein that extends in longitudinal tube direction for flow of a liquid absorbent. The tubular reactor housing further comprises a second zone for accommodation of a catalyst material, with the second zone likewise extending in longitudinal tube direction. It is a feature of the invention that the first zone and the second zone are separated by a gas-permeable separation zone, wherein the separation zone has a mechanically self-supporting structure. The invention is further characterized in that the tubular reactor housing has an aspect ratio greater than six, which means that the length of the tube is at least six times greater than the internal diameter of the tube.

The reactor of the invention enables continuous flow of absorption liquid in the immediate environment and along a catalyst material that extends longitudinally in the tubular reactor, such that a product that forms on the catalyst surface can be continuously absorbed with separation solely by the mechanically supporting separation zone. The product absorbed by the absorbent is removed continuously from the reaction zone in the region of the catalyst material, and therefore a new product can form again on account of the reestablishment of equilibrium. The reactor needs virtually no moving parts in the reaction regime, which means that it is subject to a relatively low degree of wear and permits an inexpensive technical construction. According to the flow rate of the absorbent along the first zone and according to the introduction of reactant gases into the second zone, the progression of the reaction and the level of conversion can be regulated. When small amounts of energy are available, the reaction can be run down to a minimum; in the case of a surplus of electrical energy, the reaction in the reactor can be intensified correspondingly and continuously. The process is a continuous process, controlled especially via the continuous flow of the absorbent through the first zone.

The first zone of the reactor comprises a porous structure, which in turn advantageously has a hydrophilic surface, i.e., more particularly, a hydrophilic surface with respect to the absorption liquid. What is meant here by “hydrophilic” is that the wetting angle between the surface and the corresponding liquid is less than 90 degrees. By contrast, a wetting angle exceeding 90 degrees is hydrophobic.

What is meant here by the term “tubular” is an elongate structure which is hollow on the inside and has an aspect ratio greater than six, advantageously greater than eight, more advantageously greater than twelve. The cross section of the tubular reactor housing is advantageously round or oval, although “tubular” also comprehends other cross sections, for example rectangular or square. What is meant by the term “separation zone” is a region having at least one mechanically self-supporting structure that serves more particularly to separate the catalyst material from the first zone, namely the zone through which the absorbent flows, since the mode of action of the catalyst material would be adversely affected in the event of contact with the absorbent. The separation zone itself may comprise multiple measures of a mechanical and/or chemical nature; for example, it may comprise hydrophobic layers, or spacers that keep a mechanically self-supporting mesh structure or a film structure/a membrane comprising the catalyst material spaced apart from the first zone.

In an advantageous embodiment of the reactor, the first zone is disposed on an inner wall of the tubular reactor housing. The absorption liquid can flow along the reactor housing at the inner wall, advantageously driven by gravity, and absorb reaction products as it does so that are gaseous under the ambient conditions.

Advantageously, the first zone, proceeding from the inner wall of the tubular reactor, surrounds the second zone concentrically. This means that the second zone comprising the catalyst material is disposed at the center of the tubular reactor, and this second zone is surrounded concentrically by the first zone. The term “concentrically”, in an analogous manner, also includes non-circular, i.e. even rectangular, cross sections as well. This enables a reactor construction which is industrially achievable with a low level of complexity.

In one embodiment of the invention, the first zone and the second zone extend along an entire reaction zone of the tubular reactor housing in longitudinal pipe direction and they are separated along the reaction zone by the separation zone. This elongate construction with continuous contact between first and second zone, separated merely by the separation zone, permits particularly continuous progression of the reaction and continuous passage of the absorbent through the reactor. It is appropriate here that the reactor is not in a vertical arrangement, and that it has at least an angle of 10 degrees, advantageously more, ideally 90 degrees, to the vertical. The choice of the angle of inclination of the reactor allows control of the flow rate of the absorbent, and hence an influence on the reaction per se.

A porous structure with an advantageously hydrophilic surface permits the slow uniform and controlled flow of the absorption liquid through the first zone, with the porous structure creating a maximum surface area along which the absorption medium can flow.

Moreover, in an advantageous embodiment, the mechanically self-supporting structure of the separation zone has been provided with a hydrophobic layer, which in turn has the effect that liquid absorbent is prevented from penetrating into the second zone, and hence is prevented from coming into contact with the catalyst material.

The mechanically self-supporting structure, whether in the form of a ceramic medium or in the form of a mesh or a weave, configured in such a shape that it withstands the process temperatures, has a porosity greater than the porosity of the porous structure of the first zone. This allows penetration of the separation zone by the products and subsequent absorption of the products by the absorbent.

In a further configuration of the invention, the reactor comprises an absorbent inlet and an absorbent outlet for continuous passage of the absorbent through the first zone, and comprises a reactant gas inlet for admission of the reactant gas into the second zone. More particularly, advantageously no reactant gas outlet from the second zone is provided; the reactant gas advantageously remains in a steady state in the second zone until it is converted completely. Such a construction of the second zone is also referred to as dead-end design.

A further constituent of the invention is a reactor bundle comprising at least two reactors. The reactor bundle has the feature that the at least two reactors are disposed collectively in a cooling liquid vessel.

A further constituent of the invention is a method of operating a reactor or reactor bundle, wherein the method is characterized in that an absorbent is introduced continuously into the first zone and discharged again at one end of the tubular reactor housing. In addition, the gaseous reactant is introduced into the second zone, where it reacts at least partly to give at least one product at a surface of the catalyst material present therein, i.e. in the second zone. The product thus reacted is then guided through the separation zone into the first zone and absorbed by the absorbent, and the product is discharged from the reactor with the absorbent.

Both the reactor bundle and the method of operating a reactor have the same advantages that have already been set out with regard to the reactor. More particularly, the process results in continuous progression and good controllability of the reaction with a low level of technical complexity. This is also attributable to dispensing with moving mechanical parts, which is a feature both of the method and of the reactor bundle.

It is also appropriate in one configuration to establish a combination between the reactor and a power generation unit, and hence to generate a power plant system that utilizes the described advantage of the reactor overall in order to convert surplus energy directly to chemical materials of value. This is also understood to mean using the surplus energy for generation of the reactants, for example for the operation of an electrolyzer for production of hydrogen. It is possible here with an advantage to use methanol reaction already described. The power generation unit is generally a power generator, but photovoltaic systems may also serve as power generation unit. A combination of the power generation unit and the reactor in the power plant system mentioned is appropriate especially when the power plant which comprises the power generation unit as a central unit is generating energy that would achieve too low a price on the free power market. From a particular price limit, the industrial conversion of the electrical energy to a chemical material of value is then favorable. The power plant may be of different design; it may be a plant for generation of renewable energy, for example a wind turbine; it may likewise be a conventional, especially fossil, large-scale power plant, for example a gas-fired power plant.

### BRIEF DESCRIPTION OF THE DRAWINGS

Further configurations of the invention and further features are elucidated in detail by the following figures. The figures show:

FIG. 1 a schematic diagram and detail from a reactor having a tubular housing and

FIG. 2 a reactor bundle composed of a multitude of reactors according to FIG. 1.

### DETAILED DESCRIPTION OF INVENTION

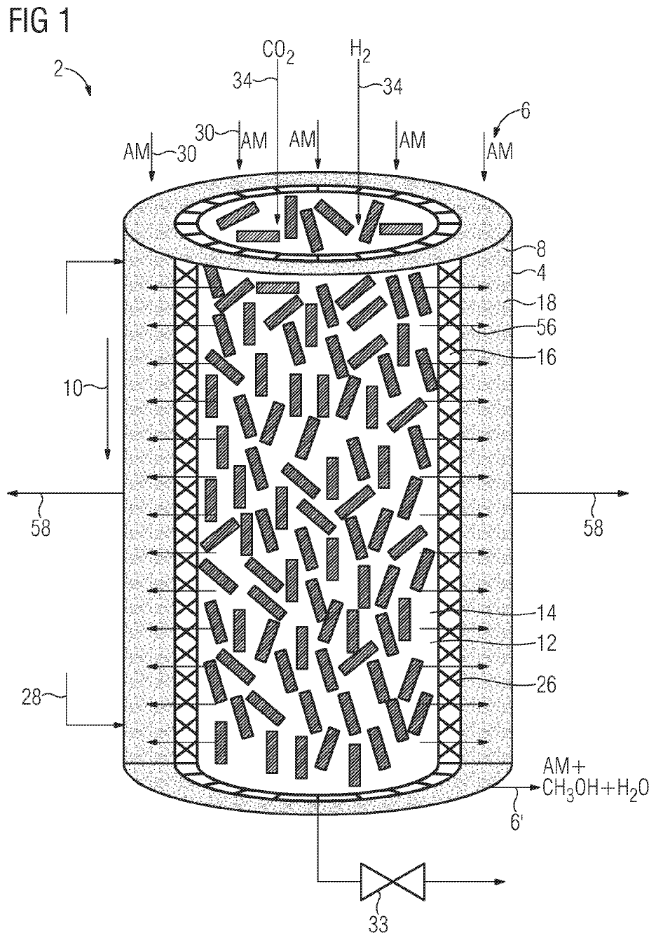

FIG. 1 shows a reactor 2 comprising a tubular reactor housing 4. The tubular reactor housing 4 accommodates a first zone 8 at an inner wall 20 of the reactor housing 4, which serves for passage of an absorbent (AM) 6. This first zone 8 advantageously has a porous structure 18 in order that the absorbent 6 comes into contact with a maximum surface area and a maximum amount of absorbent with a large surface area can be guided through the first zone 8. The reactor 2 here is upright, such that the absorbent 6 advantageously flows through the first zone 8 with the aid of gravity. The porous structure 18 increases the surface area and hence the absorption capacity of the absorbent, but it would also be favorable in principle to allow the absorbent 6 to flow gradually along the inner wall 20 of the reactor housing 4. The absorbent 6 is fed into the first zone 8 in the upper region of the reactor, which is not shown in technical detail in FIG. 1.

A second zone 12 concentrically surrounded by the first zone 8 is in the center of the reactor housing 4, with essentially a catalyst material 14 disposed in the second zone 12. There is a multitude of favorable options for configuration of the catalyst material; it is especially a bed, but it is also possible to provide a porous support material having a high surface area on which catalyst material has been applied in thin layers, similarly to an exhaust gas catalytic converter from automotive technology. A less expensive option, however, is a bed of a catalyst material, the catalyst used for methanol production being, for example, a mixture of copper, aluminum oxide and zinc oxide. The grain size and grain form of the bed material are matched here to the process technology.

It has been found that the absorbent 6 and the catalyst material 14 should have a minimum degree of contact, since the mode of action of the catalyst material 14 is otherwise restricted. For this reason, a separation zone 16 is disposed between the second zone 12 and the first zone 8, and this serves especially to prevent precisely this contact between absorbent 6 and catalyst material 14. This means that the separation zone 16 is determined especially by its function; for fulfillment of this function, it may contain multiple constituents with multiple different modes of action. The separation zone 16 here may contain, for example, a weave (not shown here), for example made of metal, such as stainless steel, or of carbon or other mineral fibers, in which the catalyst material 14 is retained. This describes one of the essential properties of the separation zone 16, that it comprises a mechanically self-supporting structure 26. The self-supporting structure 26 is thus fulfilled, for example, in the form of a thermally stable and chemically inert weave. However, it may also be configured in the form of a porous ceramic pot. What is important here is the physical and chemical separation between the absorbent 6 and the catalyst material 14. In addition, for example, spacers (not shown here explicitly) may be disposed on the inner wall 20 of the reactor housing 4, which, according to this definition, also form part of the separation zone 16, and keep the self-supporting structure 26, configured, for example, in the form of a weave, away from the first zone 8.

In the second zone 12, a gaseous reactant 54, comprising carbon dioxide and hydrogen particularly in the case of methanol production, is now introduced into the second zone 12 of the reactor 2. The reactor mentioned is a working example; alternative reactant gases for implementation of equilibrium-limited reactions are likewise appropriate here. The carbon dioxide and hydrogen react at the surface of the catalyst material 14 to give methanol, which is gaseous under the prevailing process conditions, for example 50 bar and 250 degrees Celsius. The methanol product which is gaseous (under the process conditions) diffuses through the self-supporting structure 26 into the first zone 8 and is absorbed by the absorbent 6. The absorbent 6 flowing continuously through the first zone 8 is discharged again from the tubular reactor, with the laden absorbent now given the reference numeral 6′. The laden absorbent 6′ is unloaded in an apparatus (not shown), for example by lowering the pressure, and advantageously fed back to the reaction process. The second zone 12 is advantageously closed to the reactant gas at the end of the reactor 2, i.e. at the lower end at which the laden absorbent 6′ is also discharged. This is also called a dead-end design, the effect of which is that the reactant gas introduced is forced to react completely to give the product, but further reactants 54 are introduced continuously at a reactant gas inlet 34 according to the consumption of the reactants 54. It would be technically possible, but economically disadvantageous, for reactant gases to flow through the second zone 12, which is why the dead-end design is advantageously chosen. Merely a valve 33 is provided in order to lead off what is called a purge gas from the second zone. The purge gas comprises unwanted gases, especially inert gases such as nitrogen that occur as waste products during the reaction. In this case, during the reaction, the valve 33 is opened at regular intervals.

With regard to the reactor 2 or the reactor housing 4, there should also be a definition of the longitudinal pipe direction 10 along the arrow 10 that characterizes it. In this longitudinal pipe direction 10, there is also a reaction zone 28 virtually over the entire length of the reactor housing 22. There is advantageously extension with respect to the reaction zone 28 only in the upper and lower region for discharge or supply of reactant gas 54 or input and output of the absorbent 6 in the reactor housing. This means that reaction takes place virtually over the entire length of the reactor 2, which means particularly good exploitation of space combined with inexpensive industrial implementation.

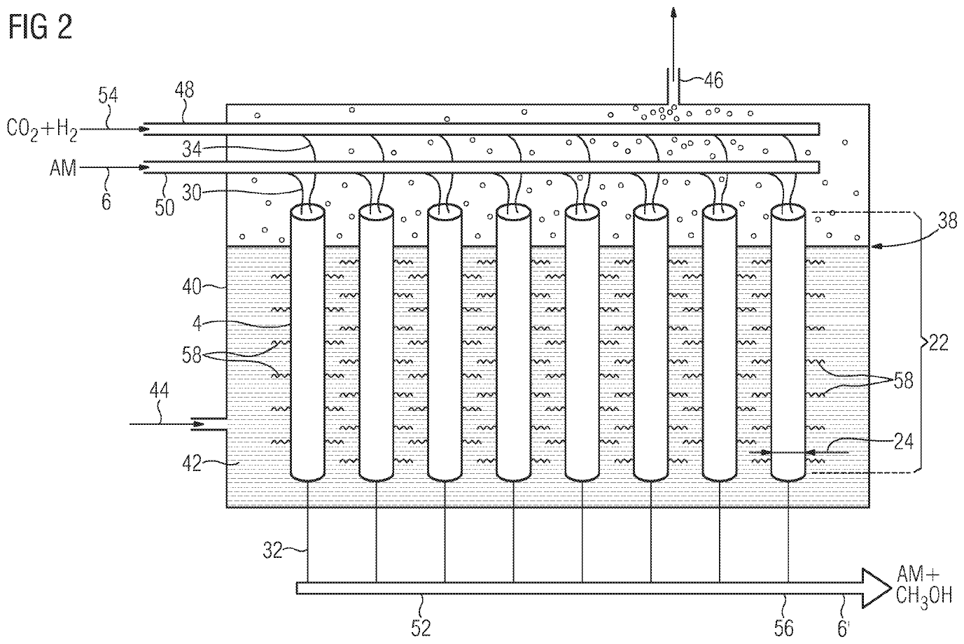

FIG. 2 shows a reactor bundle 38, wherein a multitude of reactors 2 is disposed in a cooling liquid vessel 40 in which there is likewise a cooling liquid 42. Also provided are reactant gas feeds 48 and absorbent feed 50, via which the individual reactors 2 or reactor housing 4 are respectively supplied with reactants 54 and absorbent 6. For this purpose, the reactors 2 have absorbent inlet devices 30, via which the absorbent 6 is guided into the first zone 8 of the reactor 2. In addition, the reactors have an absorbent outlet 32 in which the laden absorbent 6′ is discharged and then a product 56 is led off (not shown in detail here). Also provided is an inlet 44 for the coolant 42, wherein heat which is illustrated schematically by reference numeral 58 both in FIG. 1 and in FIG. 2 and which occurs in the reaction in reactor 2 is released to the coolant 42. The coolant 42 evaporates here and is discharged via the coolant outlet 46. This is an isothermal process regime, wherein the temperature in the reactors is kept constant by the balancing by coolant 42. The water vapor formed here is withdrawn and new coolant 42 is supplied, which contributes to the constant temperature regime.

In addition, it is appropriate when a reactor 2 or a reactor bundle 38 is combined with a power generation unit to form a power plant system. On account of the fluctuation in power supply in power grids, attributable especially to the different provision of renewable electrical energies, the cost of power changes within very short time intervals, such that it may not be possible to break even economically when feeding-in electrical energy using different power plants. In this connection, it may be appropriate for all power plant types, but conventional fossil power plants and renewable power plants, such as solar power plants or wind power plants, to cease feeding the energy generated into the power grid and instead to utilize the electrical energy generated for conversion of product gases to a chemical material of value, such as the ethanol described. As the case may be, depending on the respective cost of power and the product price to be achieved, this may mean an economic advantage. In the case of fossil power plants, it is simultaneously also possible to reduce CO2 emissions or improve the CO2 balance.

For fossil power plants too, it may be appropriate to utilize this combination between the power generation unit, especially a power generator having the described reactor 2 or the reactor bundle 38. The effect of this may be that the power plant unit, for example a gas power plant or a coal power plant, may be operated in a constant output spectrum, which is beneficial to the economic viability of the power plant. The energy generation by the power plant need not be run down when the cost of power is low; instead, the energy can be introduced, for example, into the chemical reaction described or into the reactor 2 or into the reactor bundle 38. Furthermore, especially when the technology described is applied to fossil power plants, it is appropriate to branch off carbon dioxide, which is inevitably formed in the combustion of fossil fuels, from the offgases from the power plant and to feed it into the process regime for production of gases of value, as described, as carbon dioxide reactant gas in the methanol synthesis.

### LIST OF REFERENCE NUMERALS

2 reactor
4 reactor housing
6 absorbent (AM)
8 first zone
10 longitudinal pipe direction
12 second zone
14 catalyst material
16 separation zone
18 porous structure
20 inner reactor housing wall
22 length of reactor housing
24 width of reactor housing
26 mechanically self-supporting structure
28 reaction zone
30 absorbent inlet
32 absorbent outlet
33 purge gas valve
34 reactant gas inlet
36 valve
38 reactor bundle
40 cooling fluid vessel
42 cooling fluid
44 cooling fluid inlet
46 cooling fluid outlet
48 reactant gas collection conduit
50 absorbent collection conduit
52 absorbent collection conduit outlet
54 reactants
56 product
58 heat

1.-14. (canceled)

15. A reactor for implementation of equilibrium-limited reactions, comprising:
a reactor housing in tubular configuration, comprising a first zone extending in longitudinal tube direction that serves for flow of a liquid absorbent and a second zone likewise extending in longitudinal tube direction for accommodation of a catalyst material,
wherein the first zone and the second zone are separated by a gas-permeable separation zone,
wherein the separation zone has a mechanically self-supporting structure provided with a hydrophobic layer,
wherein the aspect ratio of the tubular reactor housing along a reaction zone is greater than 6, and
wherein the first zone contains a porous structure.

16. The reactor as claimed in claim 15,
wherein the first zone is disposed at an inner wall of the tubular reactor housing.

17. The reactor as claimed in claim 15,
wherein the first zone is disposed concentrically around the inner wall of the tubular reactor housing.

18. The reactor as claimed in claim 15,
wherein the first zone concentrically surrounds the second zone, separated by the separation zone.

19. The reactor as claimed in claim 15,
wherein the first zone and the second zone extend along the entire reaction zone of the tubular reactor housing in longitudinal tube direction, separated by the separation zone.

20. The reactor as claimed in claim 15,
wherein the tubular reactor housing, positioned ready for operation, has an angle to the vertical of between 10° and 90°.

21. The reactor as claimed in claim 15,
wherein the porous structure in the first zone at least partly has a hydrophilic surface.

22. The reactor as claimed in claim 15,
wherein a porosity of the self-supporting structure is higher than a porosity of the porous structure of the first zone.

23. The reactor as claimed in claim 15,
wherein the separation zone comprises at least one hydrophobic layer.

24. The reactor as claimed in claim 15,
wherein the reactor has an absorbent inlet and an absorbent outlet for continuous passage of the absorbent through the first zone, and in that the second zone has a reactant gas inlet.

25. A reactor bundle, comprising:
at least two reactors as claimed in claim 15,
wherein the reactors are disposed collectively in a cooling liquid vessel.

26. A method of operating a reactor as claimed in claim 15, comprising:
continuously introducing a liquid absorbent into a first zone having porous structure and discharging the liquid absorbent again at one end of the tubular reactor housing, and
introducing the gaseous reactants into the second zone and wherein the gaseous reactants are at least partly reacted to give at least one product at a surface of the catalyst material present therein, and
wherein the product then arrives in the first zone through the separation zone provided with a hydrophobic layer and is absorbed by the liquid absorbent and discharged from the reactor therewith.

27. A power plant system, comprising:
a power generation unit, and
a reactor as claimed in claim 15,
wherein electrical energy generated by the power generator is utilized for operation of the reactor.
